(19) United States
(12) Patent Application Publication
(10) Pub. No.: US 2019/0255523 A1
(43) Pub. Date: Aug. 22, 2019

(54) CONCENTRATION DEVICES AND METHODS

(71) Applicant: BIOINSIGHT DIAGNOSTICS LLC, Cambridge, MA (US)

(72) Inventors: Nicholas D. Microulis, East Hampton, NY (US); Stephen T. Sonis, Wayland, MA (US)

(21) Appl. No.: 16/342,114

(22) PCT Filed: Oct. 12, 2017
(86) PCT No.: PCT/US2017/056368

§ 371 Date: Apr. 15, 2019

Related U.S. Application Data
(60) Provisional application No. 62/408,441, filed on Oct. 14, 2016.

(51) Int. Cl. B01L 3/00; A61B 10/00; G01N 21/65; G01N 33/543

(52) U.S. Cl. CPC B01L 3/5023; A61B 10/0045; A61B 10/0096; B01L 3/502715; C12Q 1/00; G01N 21/65; G01N 33/54326; B01L 3/5029; B01L 3/523

(57) ABSTRACT

A concentration device (10) includes a sample collector (3) and a main chamber (1). In some embodiments, the concentration device includes a solution chamber (4) that holds a concentrating solution that is released from the solution chamber (4) and mixes with the sample obtained by the sample collector (3). In some embodiments, the concentration device includes a microfluidic channel through which sample can move, and a pressure pump may be used to move the sample through the microfluidic channel. The concentration device can be used in performing a method of obtaining a Raman spectrum characteristic of a microbe. The concentration device can be used in performing a method of detecting and identifying a microbe in a sample.

### RELATED APPLICATIONS

This application claims the benefit of U.S. Provisional Application No. 62/408,441, filed Oct. 14, 2016, and titled “Concentration Devices and Methods,” the entirety of which is incorporated herein by reference for all purposes.

### BACKGROUND

Aspects relate generally to a concentration device and to methods of concentration and detection of microorganisms.

### SUMMARY

According to one aspect, a concentration device is provided. The concentration device includes a main chamber, a solution chamber containing a solution and a flow controller that closes fluid communication between the solution chamber and the main chamber. The concentration device also includes a sample collector.

According to another aspect, a concentration device is provided. The concentration device includes a main chamber, a pressure pump, a microfluidic channel(s) coupled to the main chamber and a sample collector.

According to another aspect, a method of obtaining a Raman spectrum characteristic of a microbe is provided. The method includes introducing a sample into a concentration device containing a concentration solution. The method includes bringing the sample into contact with the concentration solution within the concentration device. The method includes obtaining a Raman spectrum of the sample.

According to another aspect, a method of detecting and identifying a microbe in a sample is provided. The method includes introducing a sample into a concentration device containing a concentration solution. The method includes bringing the sample into contact with the concentration solution within the concentration device. The method includes obtaining a Raman spectrum of the sample.

According to another aspect, a method of obtaining a Raman spectrum characteristic of a microbe is provided. The method includes introducing a sample into a concentration device having a microfluidic channel. The method includes moving the sample into the microfluidic channel. The method includes obtaining a Raman spectrum of the sample.

According to another aspect, a method of detecting and identifying a microbe in a sample is provided. The method includes introducing a sample into a concentration device having a microfluidic channel. The method includes moving the sample into the microfluidic channel. The method includes obtaining a Raman spectrum of the sample.

### BRIEF DESCRIPTION OF DRAWINGS

Non-limiting embodiments that incorporate one or more aspects will be described by way of example with reference to the accompanying figures, which are schematic and are not necessarily intended to be drawn to scale. In the figures, each identical or nearly identical component illustrated is typically represented by a single numeral. For purposes of clarity, not every component is labeled in every figure, nor is every component of each embodiment shown where illustration is not necessary for an understanding by those of ordinary skill in the art. Various embodiments will now be described, by way of example, with reference to the accompanying drawings, in which:

FIG. 1A depicts a concentration device according to one embodiment;

FIG. 1B depicts a CAD rendering of the concentration device of FIG. 1A; FIG. 2 depicts the solution chamber shown in FIGS. 1A-1B;

FIG. 3 depicts a concentration device according to a second embodiment; FIG. 4A depicts a sample collector probe with a depressible actuator;

FIG. 4B depicts a sample collector probe with the actuator in a depressed position; FIG. 5 depicts a concentration device cooperating with a measurement instrument;

FIG. 6 depicts a flowchart of an example method of obtaining a Raman spectrum characteristic of a microbe according to one embodiment; and

FIG. 7 depicts a flowchart of an example method of obtaining a Raman spectrum characteristic of a microbe according to a second embodiment.

### DETAILED DESCRIPTION

Rapid identification of bacteria, for example at the point-of-care or food processing and storage facilities, is a highly desirable feature of diagnostics used for medical and food safety applications. For example, in the case of common sexually transmitted diseases (chlamydia and gonorrhea), current diagnostic practice entails obtaining a sample from the patient and then culturing for more than 24 hours. As a consequence of the delay between sample collection and diagnosis, an infection may worsen or patients may not return for treatment. In the case of food safety, delayed identification of potential pathogens may result in distribution and consumption of contaminated food.

The inventors have appreciated that existing methods of using Raman spectroscopy for bacterial identification required a range of steps which hindered clinical use. The inventors have recognized the need for an integrated device to capture, concentrate and stage microorganisms of interest and to interface with a measurement instrument for identification, such as a Raman spectrometer.

Disclosed herein is a device and method for concentrating microorganisms such as bacteria (e.g., E. Coli, Salmonella, Listeria, Neisseria gonorrhoeae, Chlamydia, etc.). In one aspect, the device is sized and shaped to interface with a measurement instrument to permit identification or other measurement of the concentrated microorganisms. The concentration device can be used in conjunction with the measurement instrument to perform rapid, point-of-care microorganism detection and identification based on an obtained Raman spectrum.

In some embodiments, a concentration device may permit the seamless transfer of a sample to media within the device. In some embodiments, a concentration device may introduce, from a self-contained reservoir, materials to capture specific microorganisms of interest. In some embodiments, a concentration device may contain a fixed substrate onto which the microorganisms of interest would be concentrated using either immunomagnetic or microfluidic methods. In some embodiments, a concentration device may be physically arranged to enable capture of an acquisition signal by a measurement instrument. In some embodiments, a concentration device may interface with an existing measurement instrument such as a Raman instrument. In some embodiments, the concentration device is disposable and compliant with safety requirements.

### Bead Conjugated Concentration Method

According to one aspect, microorganisms of interest in the sample may be bound to by substances from a concentrating solution when the concentrating solution is mixed with the sample within a concentration device. A tool such as an electromagnet, either external to the device (e.g. as part of a measurement instrument or other external tool), or part of the concentration device itself, may interact with the substances of the concentrating solution that are bound to the microorganisms of interest to concentrate the substances to one location of the concentration device.

In some embodiments, the concentration device may include a main chamber, a solution chamber and a sample collector. In some embodiments, the sample collector is a swab that is wiped across or otherwise physically contacted with a surface of interest to obtain a sample. The sample collector may be any suitable component used to obtain sample, such as an absorbent or adhesive material, a pipette or other device that draws fluid via suction, or any other suitable component. The composition of the probe tip may be a sponge, cotton, or other suitable material. In some embodiments, the sample collector is attached (may be removably attached) to a cap. In some cases, a shaft of the sample collector is held in place by the cap.

During use, a concentrating solution is released from the solution chamber into the main chamber and mixes with sample on and/or in the sample collector. Substances in the concentrating solution may bind with microorganisms of interest in the sample. The concentration device is then brought to a measurement instrument, which may interact with the substances of the concentrating solution that are bound to the microorganisms of interest to concentrate the substances to one location of the concentration device. In some embodiments, the concentration device includes a substrate onto which the substances and microorganisms are concentrated. In some embodiments, the measurement instrument includes a tool such as an electromagnet that causes movement of the substances to desired location.

The measurement instrument then measures a characteristic of the microorganisms at the concentrated location. In some embodiments, the main chamber of the concentration device may have a width that is sized and shaped to match the focal distance of the acquisition probe of the measurement instrument for signal acquisition by the measurement instrument.

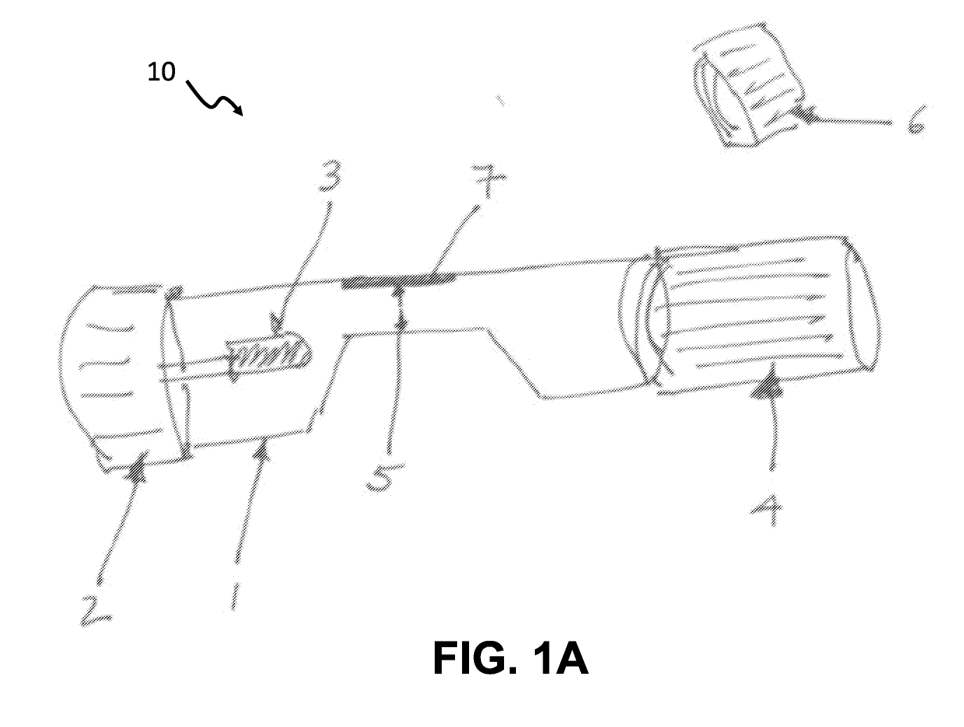

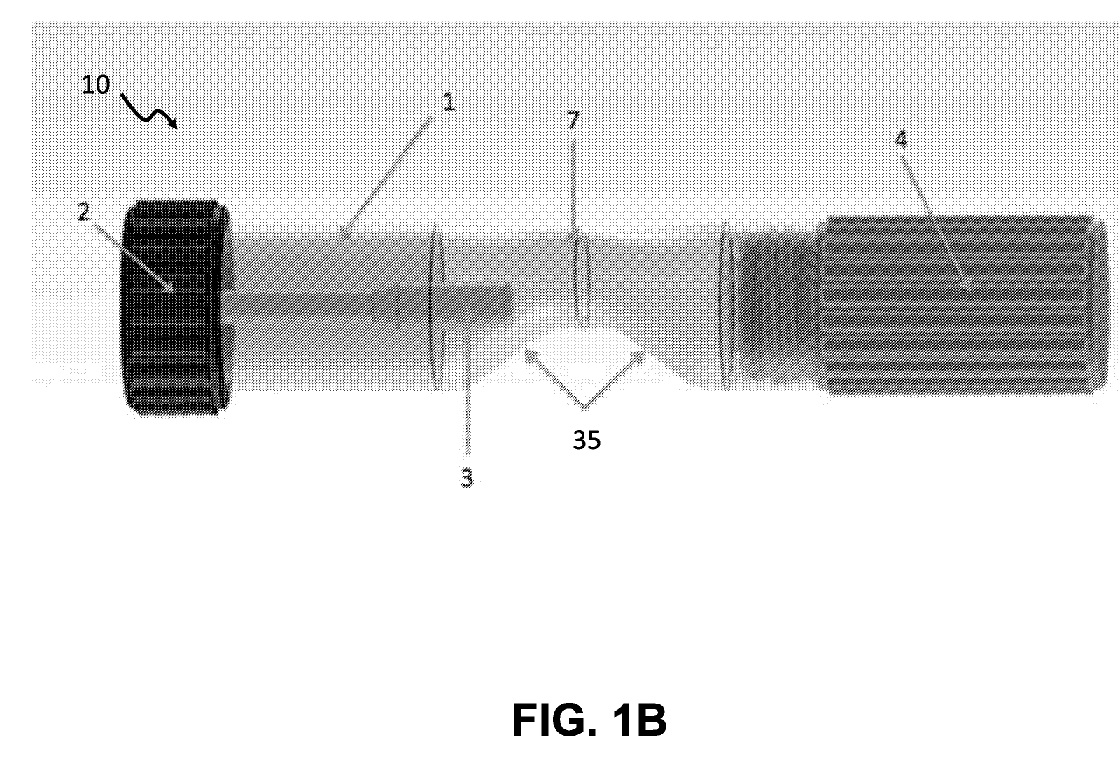

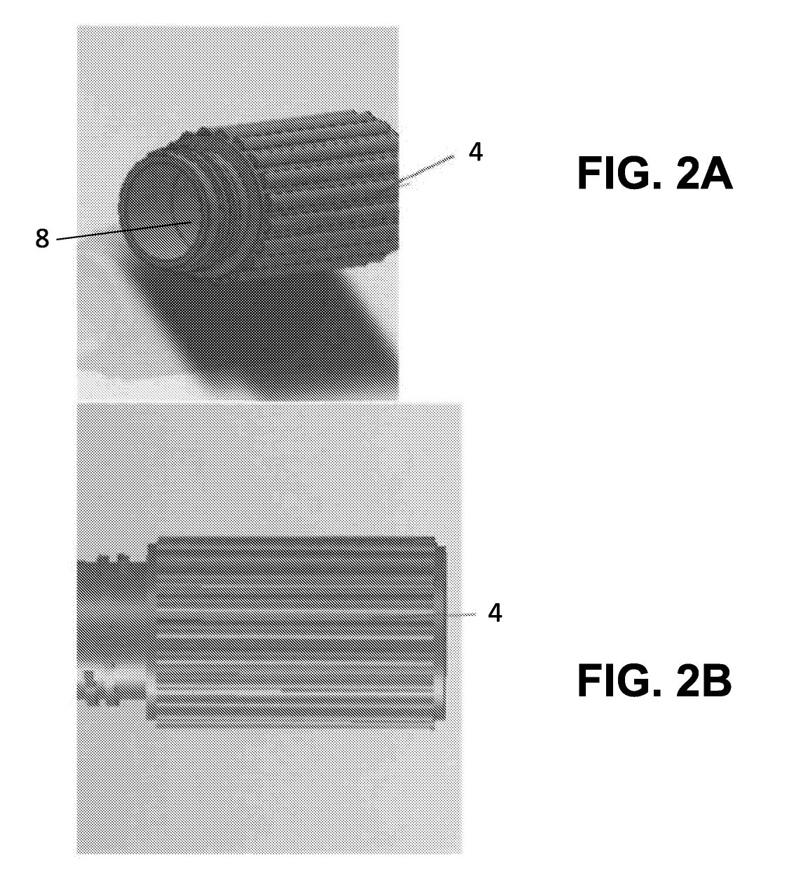

FIG. 1A depicts an illustrative embodiment of a concentration device 10. The concentration device includes a main chamber 1, a solution chamber 4 and a sample collector 3. In some embodiments, the concentration device includes a substrate 7 onto which the substances of the concentrating solution and the microorganisms of the sample are concentrated. The main chamber 1 has a width 5 that is sized to match the focal distance of an acquisition probe for signal acquisition by a measurement instrument. In some embodiments, the device is a single use disposable sample concentration cartridge.

In some embodiments, the sample collector 3 is coupled to a cap 2. The sample collector may be fixed to the cap or it may be retractable relative to the cap. The cap may be removably attachable to the main chamber of the concentration device. In some embodiments, the cap is threaded and removably attached to a threaded portion of the main chamber. The pitch of the threads may vary.

During use, the cap 2 with attached sample collector 3 is removed and used to obtain a sample. In the embodiment shown in FIG. 1A, the sample collector is a swab that is wiped across a surface of interest to obtain sample.

After obtaining a sample, the sample collector is inserted into the main chamber 1 of the concentration device. In embodiments where the sample collector is coupled to a cap, the cap may attach to the main chamber, which allows the sample collector to be secured in place.

After the sample collector has been inserted into the main chamber, the concentrating solution contained within the solution chamber 4 is released. Release of solution may be accomplished in a variety of ways. In the embodiment shown in FIG. 1A, the solution chamber is closed off by a membrane. Solution may be released by piercing the membrane, peeling off the membrane, or otherwise opening the membrane. In one embodiment, the solution chamber is moved toward a piercing element that pierces the membrane open. In the embodiment of FIG. 1A, the solution chamber 4 is rotatably coupled to the main chamber. Rotation of the solution chamber relative to the main chamber causes the solution chamber to move toward the main chamber. A piercing element such as a sharp internal flange may be located inside the main chamber, and may be fixed in place relative to the main chamber. As the solution chamber moves toward the main chamber, the membrane comes into contact with the piercing element and is pierced open. In some embodiments, the solution chamber is rotatably coupled to the main chamber via a threaded connection. In some embodiments, screwing the solution chamber clockwise relative to the main chamber causes the solution chamber to move toward the main chamber.

It should be appreciated that other types of flow controllers other than a membrane may be used to control fluid communication between the solution chamber and the main chamber. Other types of flow controllers include, for example, a valve, a movable door (e.g. slidable, rotatable), or any other suitable flow controller.

With the concentrating solution released from the solution chamber, the concentrating solution can interact with the sampled specimen on, in and/or obtained by the sample collector.

In some embodiments, the concentrating solution includes an antibody-metallic bead conjugates in which the antibody is directed against an antigen of interest from the sample. In some embodiments, the antigen of interest is a bacterial antigen.

The concentration device may be inverted several times to enhance mixture and/or dissolution of the sample in the concentrating fluid. In some embodiments, the solution chamber 4 may be removed and replaced with an inert transfer cap 6 prior to testing. The cap 6 may be different size and/or shape than the solution chamber 4 to permit the concentration device to fit with a measurement instrument.

According to one aspect, a portion of the concentration device may have a narrowed width. In some embodiments, the portion of the concentration device with narrowed a width may have opposing surfaces that are spaced from one another at a distance specified to be within the focal distance range for a specified type of signal acquisition. In some embodiments, the type of signal acquisition is a Raman signal acquisition. In the embodiment shown in FIG. 1A, the distance 5 may be 8 to 11 mm. In some embodiments, like that shown in FIGS. 1A-1B, the narrowed region is formed by an indent at only one side of the chamber. In other embodiments, however, the narrowed region is formed by indents at both sides of the chamber.

The substrate of the concentration device may be positioned at one of the opposing surfaces at the narrowed region. In embodiments in which only one side of the chamber is indented to form the narrowed region, the substrate may be located on the side of the region opposite the indented wall. The microorganism of interest may be collected on the substrate. In embodiments in which the microorganism of interest is one or more bacteria and the binding substance of the concentrating solution is an antibody-metallic bead that binds an antigen on the bacteria, a conjugated bacteria complex is collected on the substrate. In some embodiments, the substrate is made of a filter material, and may include nanobeads. The nanobeads, which may consist of gold, silver or other material, may be deposited and fixed to the filter material. In some embodiments, the substrate is approximately 0.45 μm in thickness. In some embodiments, the substrate is a Surface Enhanced Raman Spectroscopy (SERS) substrate, and may be in the shape of a disc.

FIGS. 2A-2B depict an embodiment of the solution chamber 4 shown in FIGS. 1A-1B. The solution chamber may also function as a cap. In the embodiment shown in FIGS. 2A-2B, the solution chamber 4 has external threads that are compatible with threads on the concentration device, e.g., on the main chamber of the concentration device. In other embodiments, the concentration device has internal threads. The solution chamber is hollow and initially contains a concentration solution. In its inactivated state, the solution is retained within the solution chamber by a membrane 8. The membrane may be made from any suitable material, such as plastic or aluminum. In some embodiments, the membrane may be liquid and/or air impermeable.

### Microfluidic Identification Method

According to one aspect, a microfluidic channel is used to concentrate microorganisms of interest to a measurement location. A concentrating solution may not be necessary in these embodiments. In some embodiments, the concentration device includes a microfluidic channel through which sample can flow. The device may include a pressure pump used to force movement of sample through the microfluidic channel. The pressure pump may be manual or electric. Movement of the sample through the microfluidic channel forces microorganisms of interest to pass through the channel. As the microorganisms pass through the channel, a measurement instrument cooperates with the concentration device to acquire information about the microorganisms. By virtue of the position of the microfluidic channel within the concentration device, the microorganisms passing through the microfluidic channel are positioned at a distance from the measurement instrument that matches the focal length of the measurement instrument.

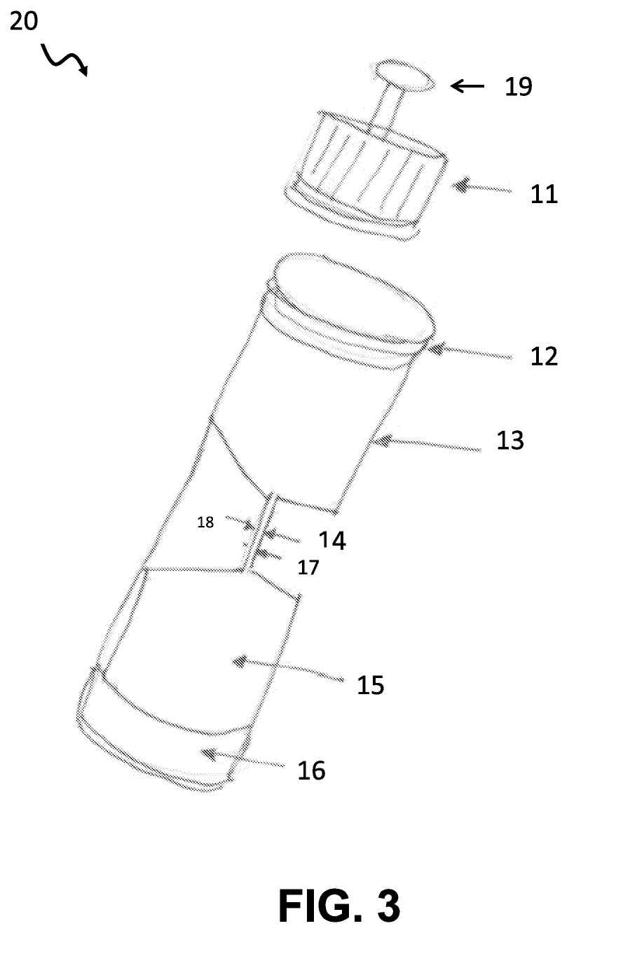

FIG. 3 depicts an illustrative embodiment of a concentration device 20 in which un-conjugated microorganisms such as bacteria are passed through a microfluidic channel 17. In some embodiments, sample is obtained and inserted into the main body of the concentration device using a sample collector as described above. In some embodiments, following sample collection, the sample collector and cap (not shown in FIG. 3; see components 2 and 3 in FIG. 1A) are removed and replaced with a different cap 11 that includes a pressure pump 19 such as a plunger. The cap may have threads that are compatible with the main chamber. The plunger cap may be connected by a plastic rod (or other suitable material) to a flat surface base so that, after the cap has been screwed back on to the tube, the plunger can be compressed to create positive pressure. An air-release element 16 may be present on the opposite end of the device to permit flow of test solution from the top half of the main chamber 13 through a microfluidic channel 17. The air-release element may be, for example, a valve such as a one-way valve. In some embodiments, the air-release element is mounted to a cap that is removably attachable to the rest of the concentration device. One side of the main chamber 13 may be compressed and flattened at the center 14 to match the focal length of the Raman probe. In some embodiments, the focal length is 8-11 mm. An opposite inner wall 18 of the device 20 may be machined to create space to collect light or other form of energy from the Raman instrument or other measurement instrument as microorganisms pass through the chamber.

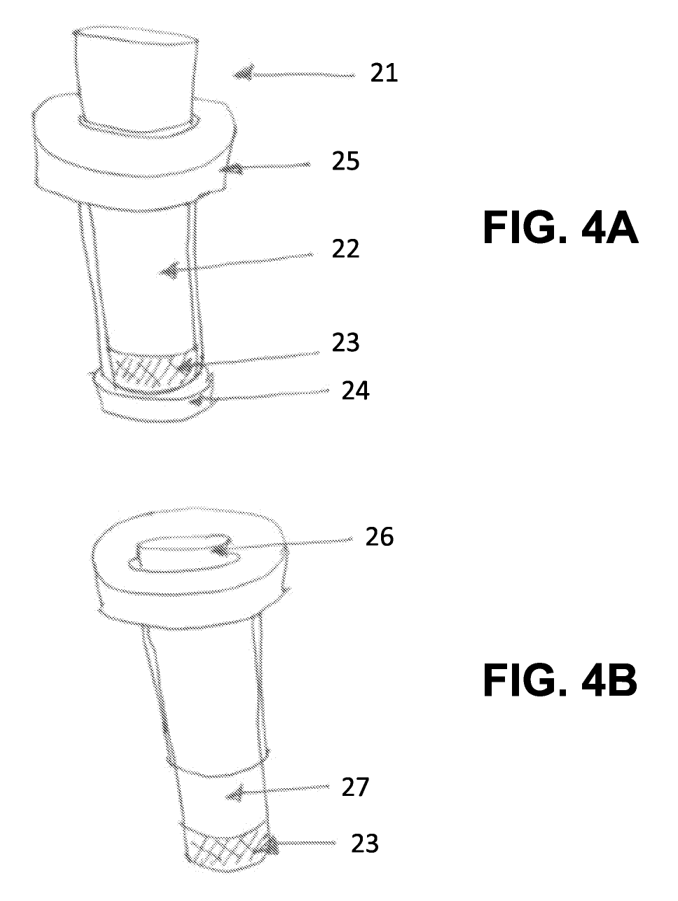

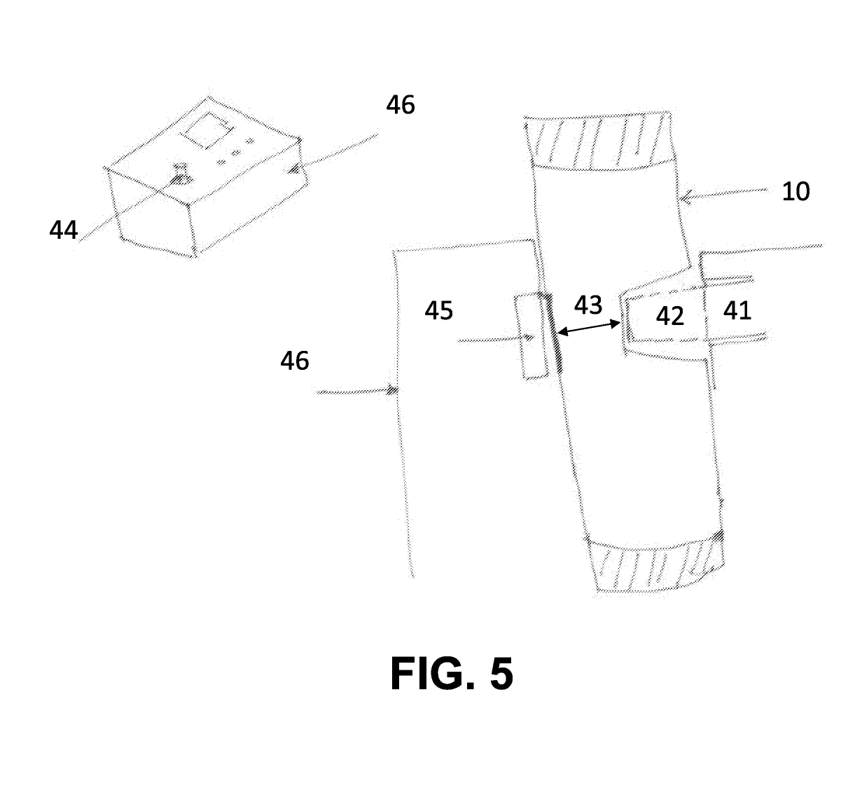

It should be appreciated that different types of sample collectors may be used. FIGS. 4A-4B depict one illustrative embodiment of a sample collector. The probe may be made of sterilizable plastic. In some embodiments, the probe includes a threaded cap 25 compatible with the concentration devices described above. In the embodiment shown in FIGS. 4A-4B, the probe consists of a movable sampling element 23. In some embodiments, the sampling element 23 is coupled to a movable inner core 22, such as a spring-loaded, depressible core. The sampling element 23 may be made of an absorbent material such as a sponge. In its resting position the core and sponge are enclosed in the outer sleeve of the device and covered with a protective, cap 24. Cap 24 may be threaded. To activate the probe, the cap 24 is removed and the actuator is moved from a pre-deployment position 21 shown in FIG. 4A to a depressed position 26 shown in FIG. 4B, thereby exposing the sampling element 23 and, in some embodiments, a portion 27 of the inner core. Following sampling, the actuator is released, and the actuator and core may return to the original pre-deployment position 21. The end of the probe is capped 24 and the sample is then ready for transfer into a device.

FIG. 5 depicts a cross-section of a concentration device 10 from FIGS. 1A-1B cooperating with a measurement instrument 46 such as a Raman signal acquisition instrument. The concentration device 10 fits with the measurement instrument 46, which may have a holder for receiving the device. When the concentration device is initially received by the measurement instrument, the acquisition probe (such as a Raman probe) may be in an ‘at rest’ position 41. In some embodiments, solution from the solution chamber may have been previously released and mixed with the sample within the concentration device prior to interaction between the concentration device and the measurement instrument. In some embodiments, the solution chamber is removed from the concentration device and replaced by a transfer cap. In some embodiments, the transfer cap has a different size and/or shape relative to the solution chamber. In some cases, the concentration device cannot physically fit with the measurement instrument when the solution chamber is attached, but can fit with the measurement instrument when the solution chamber is replaced by the transfer cap.

In some embodiments, the measurement instrument may include an electromagnet. After the concentration device is received by the measurement instrument, the instrument's electromagnet is activated to enable the microorganism antibody complex to be evenly positioned and distributed onto the substrate of the concentration device. The acquisition probe of the measurement instrument then extends to an acquisition position 42 to contact the device 10 surface to ensure that the proper focal length for signal acquisition is achieved.

As depicted in FIG. 5, the measurement instrument 46 may include an actuator 44 that may be used to activate the electromagnet and/or signal acquisition.

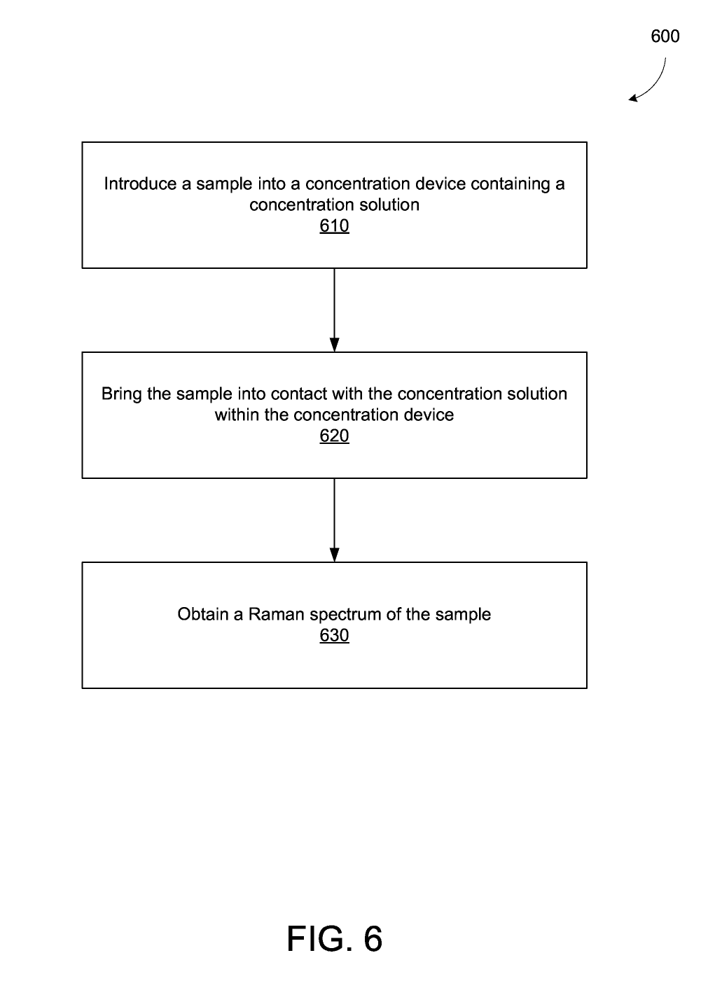

FIG. 6 depicts a flowchart of an example method 600 of obtaining a Raman spectrum characteristic of a microbe according to one embodiment. The method 600 can also be suitable for detecting and identifying a microbe in a sample. The method 600 includes introducing sample into a concentration device containing a concentration solution (stage 610). The method 600 includes bringing the sample into contact with the concentration solution within the concentration device (stage 620). The method 600 includes obtaining a Raman spectrum of the sample (stage 630).

The method 600 includes introducing sample into a concentration device containing a concentration solution (stage 610). The concentration device can be, for example, the concentration device 10 described previously. The sample can be introduced, for example, using a sample collector, such as the sample collector 3 previously described. In some implementations, the sample collector can be used to swab a substance or material to be sampled, and introduce the sample into the concentration device. The concentration device contains a concentration solution. In some implementations, the concentration solution can be contained within a solution chamber, such as the solution chamber 4 previously described. In some implementations, a membrane can seal the solution chamber and isolate the contents of the solution chamber from a main chamber of the concentration device until such time as the membrane is pierced.

The method 600 includes bringing the sample into contact with the concentration solution within the concentration device (stage 620). In some implementations, bringing the sample into contact with the concentration solution can be accomplished by piercing the membrane of the concentration device. In some implementations, the concentration solution can include a substance that will bond with a target microbe. In some implementations, the concentration solution can include an antibody-metallic bead conjugate. The antibody-metallic bead conjugate, when brought into contact with the sample, can bind an antigen on the target microbe. The antibody-metallic bead conjugate can be used to concentrate the target microbe. For example, in some implementations, an electromagnet can be used to preferentially attract the bound target microbe and conjugate. In some implementations, the concentration device can include a substrate, and the electromagnet can attract the bound target microbe and conjugate toward the substrate.

The method 600 includes obtaining the Raman spectrum of the sample (stage 630). The Raman spectrum can be obtained, for example, using the measurement instrument 46 previously described. The measurement instrument can be a Raman signal acquisition instrument such as a Raman spectrometer. The concentration device, including the sample and the concentration solution, can be inserted partially or completely into the measurement instrument. In some implementations, the method 600 can include concentrating the target microbe on a particular surface or region of the concentration device using the electromagnet. In some implementations, the concentration device can have a geometry that positions the particular surface or region of the concentration device at or near a focal point of the measurement instrument when the concentration device is inserted into the measurement instrument. In some implementations, the method 600 can include concentrating the target microbe on or near the substrate using the electromagnet. In some implementations, the concentration device can have a geometry that places the substrate at or near a focal point of the measurement instrument when the concentration device is inserted into the measurement instrument.

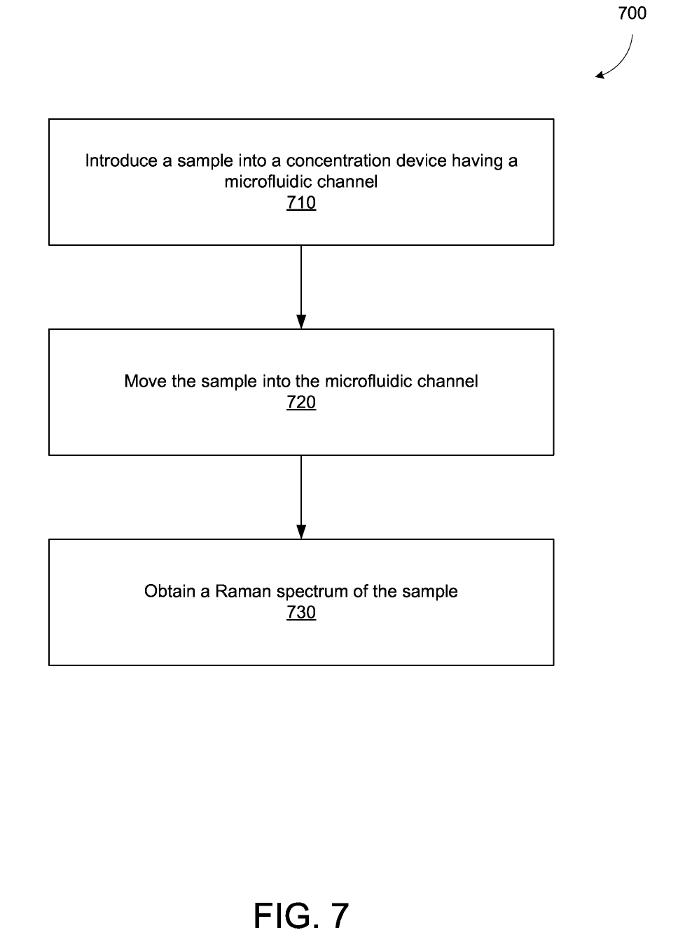

FIG. 7 depicts a flowchart of an example method 700 of obtaining a Raman spectrum characteristic of a microbe according to a second embodiment. The method 700 can also be suitable for detecting and identifying a microbe in a sample. The method 700 includes introducing a sample into a concentration device having a microfluidic channel (stage 710). The method 700 includes moving the sample into the microfluidic channel (stage 720). The method 700 includes obtaining a Raman spectrum of the sample (stage 730).

The method 700 includes introducing the sample into the concentration device having the microfluidic channel (stage 710). The concentration device can be, for example, the concentration device 20 described previously. The sample can be introduced, for example, using a sample collector, such as the example sample collectors previously described with reference to FIGS. 4A and 4B. In some implementations, the sample collector can be used to swab a substance or material to be sampled, and introduce the sample into the concentration device.

The method 700 includes moving the sample into the microfluidic channel (stage 720). The concentration device can have a pressure pump for causing a pressure differential across the microfluidic channel that will push the sample into and through the microfluidic channel. The pressure pump can include a manual or electric plunger such as those previously described. In some implementations, the concentration device can include an air-release element, such as the air-release element 16 previously described, for releasing pressure on a side of the microfluidic channel opposite the pressure pump. In some implementations, the pressure pump and/or the air-release element can be actuated to move the sample into the microfluidic channel prior to obtaining the Raman spectrum.

The method 700 includes obtaining the Raman spectrum of the sample (stage 730). The Raman spectrum can be obtained, for example, using the measurement instrument 46 previously described. The measurement instrument can be a Raman signal acquisition instrument such as a Raman spectrometer. The concentration device can be inserted partially or completely into the measurement instrument prior to obtaining the Raman spectrum. In some implementations, the concentration device can have a geometry that positions the microfluidic channel of the concentration device at or near a focal point of the measurement instrument when the concentration device is inserted into the measurement instrument.

It should be appreciated that the concentration device arrangement described here may be formed with one or more of the above-described features. The above aspects and features may be employed in any suitable combination as the present invention is not limited in this respect. It should also be appreciated that the drawings illustrate various components and features which may be incorporated into various embodiments. For simplification, some of the drawings may illustrate more than one optional feature or component. However, the invention is not limited to the specific embodiments disclosed in the drawings. It should be recognized that some embodiments may include only a portion of the components illustrated in any one drawing figure, and/or may also encompass embodiments combining components illustrated in multiple different drawing figures.

It should be understood that the foregoing description of various embodiments are intended merely to be illustrative thereof and that other embodiments, modifications, and equivalents are within the scope of the claims appended hereto.

1. A concentration device, comprising:
a main chamber;
a solution chamber containing a solution;
a flow controller that closes fluid communication between the solution chamber and the main chamber; and
a sample collector.

2. The concentration device of claim 1, wherein the flow controller comprises a membrane.

3. The concentration device of claim 1, wherein the flow controller comprises a valve.

4. The concentration device of claim 1, wherein the solution chamber is movable relative to the main chamber.

5. The concentration device of claim 4, wherein the solution chamber is rotatable relative to the main chamber.

6. The concentration device of claim 5, wherein rotation of the solution chamber relative to the main chamber causes the solution chamber to move towards the main chamber.

7. The concentration device of claim 4, wherein movement of the solution chamber relative to the main chamber causes the flow controller to open fluid communication between the solution chamber and the main chamber.

8. The concentration device of claim 7, wherein the movement comprises rotational movement.

9. The concentration device of claim 1, wherein the solution chamber is coupled to the main chamber.

10. The concentration device of claim 9, wherein the solution chamber is directly coupled to the main chamber.

11. The concentration device of claim 9, wherein the solution chamber is coupled to the main chamber via threads.

12. The concentration device of claim 1, wherein the sample collector comprises a body and a sampling element, the sampling element being movable relative to the body.

13. A concentration device, comprising:
a main chamber;
a pressure pump;
a microfluidic channel coupled to the main chamber; and
a sample collector.

14. The concentration device of claim 13, wherein the pressure pump comprises a plunger.

15. The concentration device of claim 13, wherein the pressure pump is manual.

16. The concentration device of claim 13, wherein the pressure pump is electric.

17. The concentration device of claim 13, wherein the pressure pump is removably couplable to the main chamber.

18. The concentration device of claim 13, wherein the pressure pump is interchangeable with the sample collector.

19. The concentration device of claim 13, further comprising an air-release element.

20. The concentration device of claim 19, wherein the air-release element comprises a valve.

21. A method of obtaining a Raman spectrum characteristic of a microbe, comprising:
introducing a sample into a concentration device containing a concentration solution; bringing the sample into contact with the concentration solution within the concentration device; and
obtaining a Raman spectrum of the sample.

22. A method of detecting and identifying a microbe in a sample, comprising:
introducing a sample into a concentration device containing a concentration solution; bringing the sample into contact with the concentration solution within the concentration device; and
obtaining a Raman spectrum of the sample.

23. The method of claim 21, further comprising:
inserting the concentration device into a Raman spectrometer prior to obtaining the Raman spectrum.

24. The method of any of claims 21, further comprising:
bringing the sample into contact with the concentration solution by piercing a membrane of the concentration device.

25. The method of any of claims 21, further comprising:
concentrating the microbe on a substrate of the concentration device prior to obtaining the Raman spectrum.

26. The method of claim 25, wherein the concentration solution includes an antibody-metallic bead conjugate, the method further comprising:
concentrating the microbe on the substrate using an electromagnet.

27. A method of obtaining a Raman spectrum characteristic of a microbe, comprising:
introducing a sample into a concentration device having a microfluidic channel;
moving the sample into the microfluidic channel; and
obtaining a Raman spectrum of the sample.

28. A method of detecting and identifying a microbe in a sample, comprising:
introducing a sample into a concentration device having a microfluidic channel;
moving the sample into the microfluidic channel; and
obtaining a Raman spectrum of the sample.

29. The method of claim 27, wherein the concentration device includes a pressure pump for moving the sample into the microfluidic channel.

30. The method of any of claims 29, wherein the concentration device includes an air-release element on a side of the microfluidic channel opposite the pressure pump, the method further comprising:
releasing air from the concentration device prior to obtaining the Raman spectrum.

31. The method of any of claims 30, further comprising:
inserting the concentration device into a Raman spectrometer prior to obtaining the Raman spectrum.
